(19) United States
(12) Patent Application Publication
(10) Pub. No.: US 2026/0188499 A1
(43) Pub. Date: Jul. 2, 2026

(54) METHOD OF PREDICTING OBESITY USING ENSEMBLE MODEL AND DEVICE FOR PROVIDING SOLUTION

(71) Applicant: GP CO., LTD., Gwangmyeong-si (KR)

(72) Inventors: Ji Hun KIM, Gwacheon-si (KR); Je Hyeok SEONG, Gwangmyeong-si Gyeonggi-do (KR); Do Hyun CHUN, Seoul (KR)

(21) Appl. No.: 19/460,389

(22) Filed: Jan. 27, 2026

Related U.S. Application Data
(63) Continuation of application No. PCT/KR2025/002594, filed on Feb. 25, 2025.

(30) Foreign Application Priority Data
Dec. 29, 2024 (KR) 10-2024-0191321

(51) Int. Cl. G16H 50/20; G16H 20/60; G16H 50/30; G16H 50/70

(52) U.S. Cl. CPC G16H 50/20; G16H 20/60; G16H 50/30; G16H 50/70

(57) ABSTRACT

Provided is a method of predicting obesity by ensembling obesity prediction results obtained by using different methods in order to increase the accuracy of predicting obesity in children and adolescents. A method of predicting obesity using an ensemble technique that may include receiving physical information of an evaluation subject; generating a plurality of obesity prediction values through different neural networks trained based on the received physical information; predicting obesity by comparing the plurality of obesity prediction values; and generating different solutions according to a difference between the plurality of obesity prediction values.

### TECHNICAL FIELD

The present invention relates to a method of predicting obesity of children and adolescents based on obesity prediction results obtained using different methods, and a device for providing a solution therefor.

### BACKGROUND ART

Recently, the development of artificial intelligence technology has led to its application in various fields, and methods for extracting inherent features from data through a neural network model instead of existing data processing schemes to generate additional information have been developed and used.

A neural network model used for artificial intelligence can detect and recognize features from input data through learning more quickly and accurately than general data processing. Recently, artificial intelligence technology is being applied not only to simple tracking and detection of objects but also to learning past histories and deriving current features that reflect future predictions or time-series changes.

Here, predictive analysis is a technology in the field of statistics and data mining for extracting information from data and using the information to predict trends, behavioral patterns, and the like. Such predictive analysis may be applied to all fields necessary for decision making based on the information obtained from the data. The core of predictive analysis lies in understanding relationships between variables and then predicting unknown variables.

To this end, various approaches are used depending on data characteristics and prediction targets.

Various fields in which predictive analysis is required include the field of physical growth of children and adolescents. There is great interest among parents, adolescents, and the like in when growth in height of the children and adolescents occurs and how much growth will take place or whether there is a risk that precocious puberty, obesity, and the like that may be involved in the growth of the children and adolescents will occur.

For conventional prediction of the growth in height, a method of predicting the growth in height using an X-ray of growth plates or analyzing relationships with genetic or environmental factors has been presented (Patent Nos. 10-2075743 and 10-1866208), and a method of transforming physical data of sample subjects, whose measurement times or numbers of measurements differ, into a form suitable for training of a growth prediction model has been presented (Patent No. 10-2198302).

Although methods for predicting physical growth of children and adolescents have been presented in the related art, research on methods for predicting the risk of occurrence of precocious puberty, obesity, and the like that may be involved in the growth of children and adolescents as described above and providing corresponding solutions has been insufficient.

### DISCLOSURE

### Technical Problem

The present invention is directed to providing a method of predicting obesity by ensembling obesity prediction results obtained by using different methods in order to increase the accuracy of obesity prediction of children and adolescents.

Further, the present invention is directed to providing an optimal solution to children and adolescents by utilizing obesity prediction results obtained by using different methods.

### Technical Solution

According to another aspect, there is provided a method of predicting obesity using an ensemble technique, including: receiving physical information of an evaluation subject; generating a plurality of obesity prediction values through different neural networks trained based on the received physical information; predicting obesity by comparing the plurality of obesity prediction values; and generating different solutions according to a difference between the plurality of obesity prediction values.

The plurality of obesity prediction values may include a first obesity prediction value generated based on weight and height information of the evaluation subject; and a second obesity prediction value generated based on information including growth stage information of the evaluation subject.

The physical information of the evaluation subject may be received to generate the first obesity prediction value and the second obesity prediction value.

The second obesity prediction value may be generated based on information further including the weight and height information of the evaluation subject.

The second obesity prediction value may be generated based on information including the weight and height information of the evaluation subject input to generate the first obesity prediction value.

The generating of the solutions may include receiving the first obesity prediction value and the second obesity prediction value; and comparing a difference between the first obesity prediction value and the second obesity prediction value with a preset reference.

The generating of the solutions may further include a first solution generation step of generating a solution for reducing a weight of the evaluation subject when a difference between the first obesity prediction value and the second obesity prediction value is equal to or less than the preset value in the comparison step.

The generating of the solutions may further include generating a growth graph based on the second obesity prediction value when a difference between the first obesity prediction value and the second obesity prediction value exceeds the preset value in the comparison step.

The generating of the solutions may further include a second solution generation step of comparing information on the growth graph generated based on the second obesity prediction value with the received physical information of the evaluation subject to generate a solution.

The second solution generation step may include comparing the growth graph generated based on the second obesity prediction value with the physical information of the evaluation subject at a point in time at which the physical information of the evaluation subject is input.

The second solution generation step may include providing a solution for increasing a growth prediction value in a rapid growth stage among growth stages when a difference between the information on the growth graph generated based on the second obesity prediction value and the physical information of the evaluation subject exceeds a preset value.

The second solution generation step may include providing a solution for increasing a duration of a rapid growth stage among growth stages when the difference between the information on the growth graph generated based on the second obesity prediction value and the physical information of the evaluation subject is equal to or less than the preset value.

In the second solution generation step, the physical information of the evaluation subject that is a comparison target may be a body mass index.

According to another aspect, there is provided a program stored in a computer-readable recording medium, the program including program code for executing the method of predicting obesity using an ensemble technique described above.

According to yet another aspect, there is provided a computer-readable recording medium including program code for executing the method of predicting obesity using an ensemble technique described above.

According to yet another aspect, there is provided a device for predicting obesity using an ensemble technique and providing a solution, including: an input unit configured to receive physical information of an evaluation subject; an obesity prediction unit configured to generate a plurality of obesity prediction values based on the physical information of the evaluation subject input to the input unit; and a solution generation unit configured to generate an obesity management solution based on the physical information of the evaluation subject when the evaluation subject corresponds to obese, wherein the solution generation unit generates different solutions according to a difference between the plurality of obesity prediction values.

The obesity prediction unit may include a first prediction unit configured to generate a first obesity prediction value based on weight and height information of the evaluation subject; and a second prediction unit configured to generate a second obesity prediction value generated based on information including growth stage information of the evaluation subject.

The obesity prediction unit further includes a third prediction unit configured to generate an obesity prediction value based on an ensemble of the first prediction unit and the second prediction unit.

The third prediction unit may calculate a difference between the first obesity prediction value and the second obesity prediction value.

The solution generation unit may compare a difference between the first obesity prediction value and the second obesity prediction value with a preset reference to generate different solutions.

The respective features of the embodiments described above may be implemented in combination in other embodiments unless such features are contradictory or mutually exclusive.

### Advantageous Effects

According to various embodiments of the present invention, it is possible to accurately predict obesity based on growth stages and body composition information of children and adolescents by comparing different obesity prediction values.

It is also possible to provide an appropriate solution for preventing obesity in consideration of body composition information, growth stages, and the like of an evaluation subject.

The effects of the present invention are not limited to the above-described effects, and other effects that are not mentioned will be clearly recognized by those skilled in the art from the description below.

### DESCRIPTION OF DRAWINGS

FIG. 1 is a diagram illustrating an ensemble model according to an exemplary embodiment of the present invention.

FIGS. 2 and 3 are diagrams illustrating configurations of a method of predicting obesity and a device for providing a solution according to an exemplary embodiment of the present invention.

FIG. 4 is a diagram illustrating a predicted height and a target height for each growth stage according to an exemplary embodiment of the present invention.

FIG. 5 is a diagram illustrating the obesity prediction method according to the exemplary embodiment of the present invention.

FIG. 6 is a diagram illustrating a flow of generating a plurality of obesity prediction values according to an exemplary embodiment of the present invention.

FIG. 7 is a diagram illustrating identification information and body composition information of an evaluation subject according to an exemplary embodiment of the present invention.

FIG. 8 is a diagram illustrating a solution providing method according to an exemplary embodiment of the present invention.

FIGS. 9 to 11 are diagrams expressing, on a graph, a method of predicting obesity of an evaluation subject and providing a solution according to an exemplary embodiment of the present invention.

FIG. 12 is a diagram illustrating an obesity prediction method according to an exemplary embodiment of the present invention.

FIGS. 13 and 14 are diagrams illustrating a weight change by sex according to an exemplary embodiment of the present invention.

FIG. 15 is a diagram illustrating a configuration of a neural network according to an exemplary embodiment of the present invention.

FIG. 16 is a diagram illustrating a first neural network model according to an exemplary embodiment of the present invention. and

FIG. 17 is a diagram illustrating a second neural network model according to an exemplary embodiment of the present invention.

### MODES OF THE INVENTION

Hereinafter, specific embodiments of the present invention will be described with reference to the drawings. The following detailed description is provided to facilitate a comprehensive understanding of the methods, devices, and/or systems described in the present specification. However, this is merely an example, and the present invention is not limited thereto.

When it is determined that detailed description of known technologies related to the present invention may unnecessarily obscure the gist of the present invention in describing the embodiments of the present invention, the detailed description will be omitted. The terms to be described below are terms defined in consideration of functions in the present invention and may vary depending on, for example, a user's or operator's intention or practice. Therefore, the definitions should be made based on the overall content of the present specification.

The terms used in the detailed description are merely for describing embodiments of the present invention and should not be construed as limiting. Unless clearly used otherwise, expressions in singular forms include plural forms.

In the present description, expressions such as “including” or “comprising” are intended to indicate certain features, numerals, steps, operations, elements, some thereof, or combinations thereof, and should not be construed to exclude the presence or possibility of one or more other features, numerals, steps, operations, elements, some thereof, or combinations thereof in addition to those described.

Terms such as first, second, A, B, (a), and (b) may be used in describing components of the embodiments of the present invention. These terms are only for distinguishing the components from each other and do not limit the nature, order, or sequence of the components.

In an exemplary embodiment of the present invention, children and adolescents may be understood as having concepts including a period during which a human body grows. More specifically, the infants, children, and adolescents that refer to evaluation subjects in the exemplary embodiment of the present invention are defined hereinafter through the meanings of baby, child, adolescent, toddler, and infant.

The infant is a person in a period continuing from a neonatal period and is a period up to two years after birth during which a person grows while holding a mother's nipple in the mouth, and infancy is a period in which all experiences of nutrition, caress, and excretion influence subsequent general tendencies, and the child is a term that usually refers to a person belonging to an age group of 6 years or older and under 13 and may also include a toddler (aged from 1 to 5 years) in a broad sense.

The adolescent is a person in an intermediate period between childhood and adulthood and generally refers to a person aged 13 years or older and under 19 years. The toddler may refer to a person from one year after birth to age 5. The child may generally refer to a person up to age 15.

Therefore, the children and adolescents that are the sample subjects may, in a narrow sense, refer to persons in a period from age 5 to age 19, including children and adolescents, and may indicate a period including all of general periods in which physical growth occurs, including from infancy to adolescence, in a broad sense.

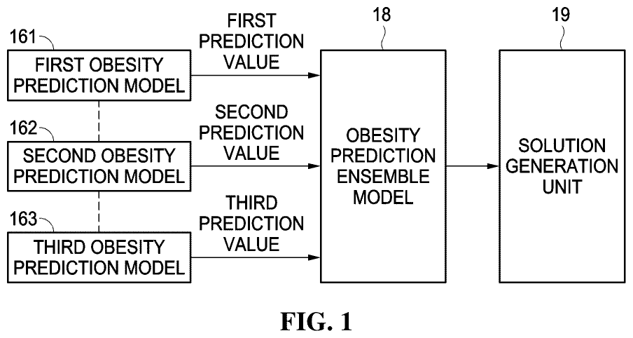

FIG. 1 is a diagram illustrating an ensemble model according to an exemplary embodiment of the present invention.

Hereinafter, a description will be given with reference to FIG. 1.

A first obesity prediction model 161 is a first model for predicting whether obesity occurs, and may mainly operate based on quantitative data such as physical measurement data (for example, weight, height, and BMI). The first obesity prediction model 161 may predict an obesity possibility using an algorithm such as linear regression or a decision tree, and ultimately output a quantitative numerical value in the form of a first prediction value.

A second obesity prediction model 162 may be defined as a model that performs elaborate prediction by reflecting complex data compared to the first obesity prediction model 161. The second obesity prediction model 162 may utilize unstructured and multivariate data such as lifestyle (dietary habits and amount of exercise) and genetic information, and may be trained through a nonlinear algorithm such as a random forest or support vector machine (SVM).

The second obesity prediction model 162 predicts whether obesity occurs from a different viewpoint than the first obesity prediction model 161, and ultimately outputs a second prediction value. The second prediction value may be used to supplementally evaluate an obesity risk level through additional data analysis.

A third obesity prediction model 163 may be defined as a model that performs more elaborate obesity prediction by using deep learning technology. The third obesity prediction model 163 may utilize, as input data, a change in health state over time (time-series data), data collected from a wearable device, and the like.

In particular, a deep learning model such as a long short term memory (LSTM) or convolutional neural network (CNN) may be applied, so that even complex and nonlinear patterns can be analyzed. The third obesity prediction model 163 ultimately generates a third prediction value, and can predict an obesity risk level with higher accuracy based on advanced data analysis.

An ensemble model 18 may serve to generate an optimal prediction result by combining prediction values derived from the first, second, and third obesity prediction models 161, 162, and 163. The ensemble model 18 may receive the respective prediction values as inputs and derive a final prediction value through various ensemble techniques.

In the present embodiment, as ensemble techniques, a scheme of assigning weights in proportion to reliability of each model through a weighted average method, a scheme of performing a combination by utilizing a meta model (logistic regression or a neural network) through a stacking technique, a scheme of combining prediction values by a majority rule through a voting-based method, and the like may be applied, and ultimately, the ensemble model may output a final obesity prediction value based on comprehensive analysis results, and this value may be delivered to a solution generation unit 19.

The solution generation unit 19 is configured to generate a customized solution for preventing the obesity based on the final obesity prediction value derived from the ensemble model 18. The solution generation unit 19 may evaluate an obesity risk level by analyzing the final prediction value, and then design the customized solution.

Examples of the solution may include a solution regarding nutrition of the evaluation subject, a solution regarding exercise, a solution regarding habit improvement, and a solution from an observation viewpoint.

The solution regarding nutrition may provide an optimized diet and nutrition intake plan based on an obesity risk level and dietary habit data of an individual, the solution regarding exercise may design an exercise program suited to a physical condition and a lifestyle pattern of an individual, the solution regarding habit improvement may present a lifestyle habit improvement scheme such as sleep management and stress management, and the solution from an observation viewpoint may apply at least one of the above-described solutions, continuously collect data of the evaluation subject, and evaluate an effect to improve the solution.

A process of providing obesity prediction and a solution through the above-described configurations will be described by way of example as follows.

First, when physical data, lifestyle, genetic information, and time-series data are input from the evaluation subject, the first obesity prediction model 161, the second obesity prediction model 162, and the third obesity prediction model 163 may independently generate a first prediction value, a second prediction value, and a third prediction value using respective data.

The derived first to third prediction values are input to the ensemble model 18, and the ensemble model may output a final optimized obesity prediction value through various ensemble techniques. The final prediction value is delivered to the solution generation unit 19, which may provide a customized obesity prevention solution based on the final prediction value.

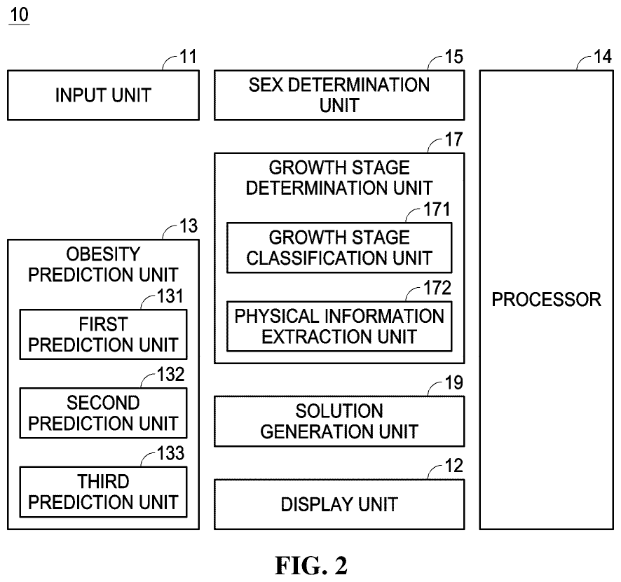

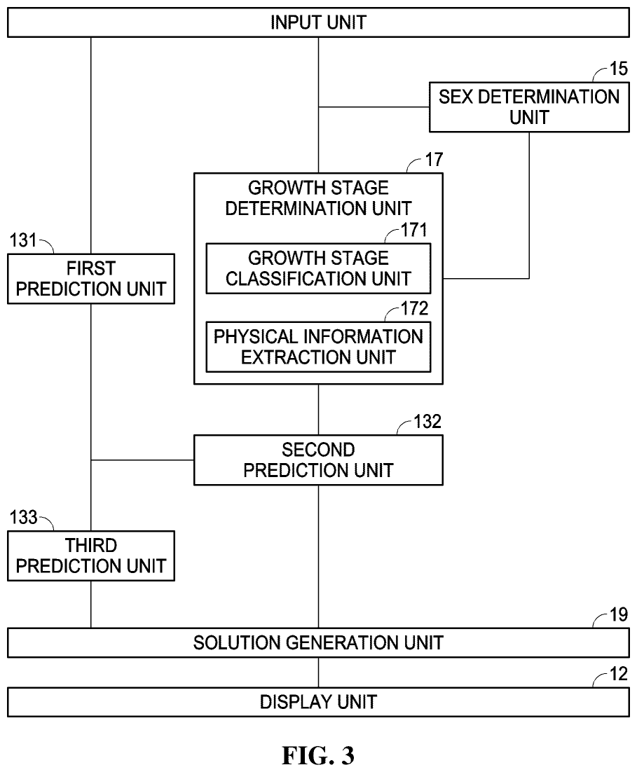

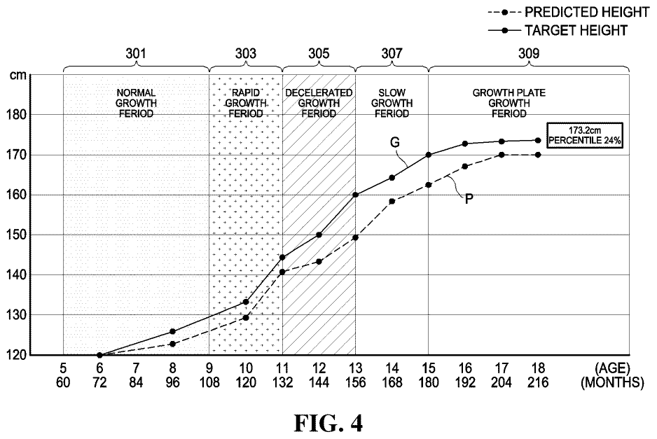

FIGS. 2 and 3 are diagrams illustrating configurations of a method of predicting obesity and a device for providing a solution according to an exemplary embodiment of the present invention, and FIG. 4 is a diagram illustrating a predicted height and a target height for each growth stage according to an exemplary embodiment of the present invention.

Hereinafter, a description will be given with reference to FIGS. 2 to 4.

A device 10 for predicting obesity using an ensemble model and providing a solution according to an exemplary embodiment of the present invention may include an input unit 11, a display unit 12, an obesity prediction unit 13, a processor 14, a sex determination unit 15, a growth stage determination unit 17, and the solution generation unit 19, the obesity prediction unit 13 may include a first prediction unit 131, a second prediction unit 132, and a third prediction unit 133, and the growth stage determination unit 17 may include a growth stage classification unit 171 and a physical information extraction unit 172.

The device 10 for predicting obesity using an ensemble model and providing a solution may receive time-series physical information of the evaluation subject through the input unit 11. The physical information of the evaluation subject may include not only basic information such as school grade (or age), sex, and height, but also additional information such as weight, protein, mineral content, body fat, body water, soft lean mass, body fat mass (fat-free mass), bone tissue, skeletal muscle mass, body mass index (BMI), basal metabolic rate, neck circumference, chest circumference, abdominal circumference, thigh circumference, arm circumference, and hip circumference. Such physical information is merely one example to aid understanding of the present invention, and the present embodiment is not limited thereto, and it is obvious that the types of information constituting the physical information may vary according to the embodiment.

The time-series physical information of the evaluation subject may be continuous information or discontinuous information. That is, the physical information of the evaluation subject may be information included in at least one of the periods corresponding to the growth stages in FIG. 2.

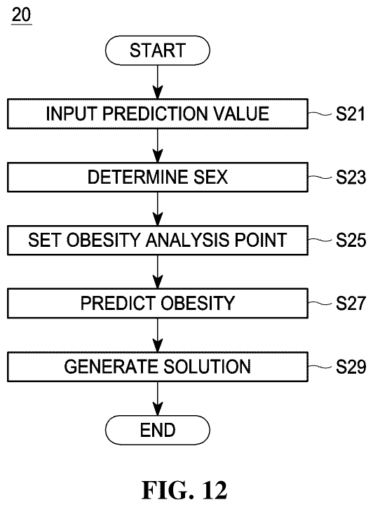

More specifically, the time-series physical information of the evaluation subject may be collected at various collection times and collection frequencies. For example, in the case of a first evaluation subject, there may be physical information measured during 8 to 12 years of age which is a portion of childhood and adolescence, and in the case of a second evaluation subject, there may be physical information measured irregularly such as at ages 8, 10 to 12, and 15. Further, in the case of a third evaluation subject, there may be physical information measured several times within a certain period (within a duration of any one growth stage among a plurality of growth stages in FIG. 12), whereas in the case of a fourth evaluation subject, there may be physical information measured only once within the certain period (within the duration of any one growth stage among the plurality of growth stages in FIG. 12).

As described above, the physical information of the evaluation subject may be included in two or more of the growth stages in FIG. 12 depending on the collection time and collection frequency (the first evaluation subject and the second evaluation subject), but there may also be cases in which that is not the case (the third evaluation subject and the fourth evaluation subject).

When there may be physical information measured several times within the duration of any one growth stage among the plurality of growth stages as in the third evaluation subject, the growth stage determination unit 17 may classify the growth stage to which the physical information of the third evaluation subject corresponds through the growth stage classification unit 171, and extract the physical information through the physical information extraction unit 172, and the extracted physical information may generally include all pieces of physical information of the evaluation subject input through the input unit 11.

However, when the physical information of the evaluation subject corresponds only to any one of the plurality of growth stages and is physical information measured only once within the period as in the fourth evaluation subject, physical information corresponding to an arbitrary period may be further generated before the physical information of the evaluation subject is input to the growth stage determination unit 17.

More specifically, the time-series physical information of the evaluation subject corresponding to the arbitrary period may be generated based on the received physical information of the evaluation subject and previously stored time-series physical growth information of a plurality of sample subjects.

As one example, the physical information may be generated based on a distribution model (similarity) between the received physical information of the evaluation subject and the previously stored time-series physical growth information of the plurality of sample subjects, or may be generated based on a Bayesian inference model (conditional probability).

The growth stage determination unit 17 may classify the growth stage into any one of the plurality of growth stages based on the physical information based on the physical information of the evaluation subject input through the input unit 11 using the growth stage classification unit 171, and extract physical information corresponding to the classified growth stage using the physical information extraction unit 172.

Referring to FIG. 4, the growth stages will be described, and the childhood and adolescence may include a normal growth period 301, a rapid growth period 303, a decelerated growth period 305, and a growth plate closure period 307.

Each growth stage may be classified according to the degree of growth, annual growth in height differs depending on each growth stage, and actual growth in height may differ depending on a growth type even within the same growth stage.

The normal growth period 301 is a period before puberty in which secondary sexual characteristics normally appear, children and adolescents corresponding to this period generally have open growth plates, and in some growth environments, the height generally increases by about 4 to 5 cm per year in a small height growth type, and the height increases in a range of about 6 to 7 cm per year in a large height growth type.

The rapid growth period 303 is a period when secondary sexual characteristics begin to appear, and in the case of females, breasts swell and breast buds form, and in the case of males, testes enlarge, pubic hair begins to grow, and a change in the voice, which is called pubertal voice change. The rapid growth period 303 generally continues for about two to three years after the normal growth period 301, and growth occurs in a range of about 7 to 10 cm per year on average.

The decelerated growth period 305 is a period in which the secondary sexual characteristics are fully developed. In this period, in females, distinction may be made based on the onset of menarche, and in males, the change can be clearly seen based on pubic hair, voice change, and axillary hair. When the decelerated growth period 305 begins, a degree of growth rapidly decreases compared to the rapid growth period 303, and the decelerated growth period 305 generally lasts about two to three years, in which growth occurs at an average rate of about 5 to 6 cm per year, and then naturally ceases. Growth plates begin to gradually close after the rapid growth period 303, and about 50% are closed about six months after entering the decelerated growth period 305.

The growth plate closure period 307 is a period in which the growth period has completely ended, but natural growth in height has become difficult, and is a period in which growth plates have closed. Generally, girls enter the growth plate closure period 307 about one year six months to two years after menarche, and boys enter the growth plate closure period 307 about one year six months to two years after a point in time when axillary hair appears. In the growth plate closure period 307, growth plates are closed and natural growth stops, but growth may still occur in a range of about 1 to 3 cm by improving physical functions through a change in poor lifestyle habits, customized exercise, posture correction, and nutrient intake.

In FIG. 4, an x-axis indicates age in months, and a y-axis indicates height (cm). The lower dotted line P indicates predicted growth of the evaluation subject, and the upper solid line G indicates target growth of the evaluation subject.

Obesity is not a simple increase in weight, but a disease involving overweight due to excessive accumulation of fat tissue in a body or a metabolic disorder due to the overweight. Childhood and adolescent obesity medically generally refers to a case in which a weight is 20% or more than the recommended weight with respect to height in an age group from infancy to puberty.

Obesity in infants mostly disappears after the first birthday as toddlers become more active and move more often. However, some obesity continues, and in many cases, obesity first returns to a normal state and then recurs during school age.

75% to 80% of childhood and adolescent obesity progresses to adult obesity. Further, obesity inhibits secretion of growth hormones, and in particular, advances puberty and shortens a growth period in the case of girls, thereby hindering growth or causing precocious puberty.

Therefore, for school-age children or adolescents who are likely to have obesity, it is necessary to predict obesity and provide a systematic solution for preventing obesity when obesity is predicted.

Meanwhile, childhood and adolescent obesity may be divided into simple obesity for which a clear cause is not identified and symptomatic obesity occurring due to a specific causative disease, and 99% or more of childhood obesity is simple obesity. Children with simple obesity generally, in both males and females, tend to have an average or slightly greater height than the plurality of sample subjects in the same age group during the normal growth period 301, and have a shorter height or a lower degree of growth than the plurality of sample subjects in the same age group after the rapid growth period 303.

In summary, childhood and adolescent obesity may be understood as a disease group involving overweight caused by various causes and s metabolic disorder due to the overweight, and in general, both males and females tend to exhibit an average or slightly higher degree of growth than the plurality of sample subjects in the same age group during the normal growth period 301, and exhibit a lower degree of growth than the plurality of sample subjects in the same age group after the rapid growth period 303.

Accordingly, in the present embodiment, for more accurate obesity prediction and solution generation, sex classification is performed through the sex determination unit 15 based on the physical information of the evaluation subject input through the input unit 11, the growth stage is classified in the growth stage determination unit 17 based on the classified sex, physical information is extracted, and then a solution may be generated in the solution generation unit 19 in consideration of the sex and growth stage of the evaluation subject.

The obesity prediction unit 13, as a type of prediction model, may be implemented as artificial intelligence with a recurrent neural network (RNN) structure so that time-series values as well as current values can be used. For example, the prediction model may be implemented with an architecture such as a long short term memory (LSTM) or a gated recurrent unit (GRU), which are recurrent neural networks. It is obvious that various conventional other artificial intelligence architectures can be applied to the prediction model of the present embodiment, the respective prediction values are derived through the plurality of prediction units 131, 132, and 133 as described above, and the solution may be generated through the processor 14 and the solution generation unit 19 based on the derived prediction values and displayed on the display unit 12.

The solution generation unit 19 may generate a growth management solution based on the physical information of the evaluation subject corresponding to the classified growth stage.

More specifically, when the evaluation subject corresponds to a normal growth stage 301, a solution for increasing a growth prediction value of the evaluation subject may be provided. The growth prediction value may be a value corresponding to the y-axis in FIG. 4, and various solutions for increasing a growth prediction value may be provided to the evaluation subject through the display unit 12 for increasing an expected target value of the y-axis.

Also, the solution generation unit 19 may compare the plurality of prediction values derived in the obesity prediction unit 13 and generate different solutions depending on the situation. This will be described in more detail through the obesity prediction method to be described later.

Meanwhile, examples of the solution provided through the display unit 120 may include current height, predicted height, degree of obesity, body fat mass, skeletal muscle mass, protein mass, mineral mass, amount of sleep, amount of exercise, nutrition information, lifestyle, posture, and the like. Each indicator may be represented as caution, normal, or good by stage with reference to a preset range, or may be represented by levels.

A current state, customized solution, precautions, and the like for each indicator may be displayed. The current state may be displayed by stage or level based on a target value, and in the case of customized solutions, content for adjusting protein, total mineral content, body fat, body water, soft lean mass, body fat mass (fat-free mass), bone tissue, skeletal muscle mass, BMI, basal metabolic rate, and the like to reach a current target value based on the received physical information may be included.

In the case of precautions, content for adjusting currently insufficient protein, total mineral content, body fat, body water, soft lean mass, body fat mass (fat-free mass), bone tissue, skeletal muscle mass, BMI, basal metabolic rate, and the like based on the received physical information may be included.

Also, when the evaluation subject corresponds to the rapid growth stage 303, the solution for increasing a growth prediction value of the evaluation subject in the rapid growth stage 303 may be provided. The growth prediction value is a value corresponding to the y-axis in FIG. 4, and as the solution for increasing a growth prediction value, various solutions for increasing an expected target value of the y-axis may be provided to the evaluation subject through the display unit 12.

Hereinafter, the interconnection of configurations of the device 10 for predicting obesity using an ensemble model and providing a solution according to an exemplary embodiment of the present invention will be described with reference to FIG. 3.

The first prediction unit 131 may receive weight and height information of the evaluation subject from the input unit 11 and generate a first obesity prediction value, and as an example, the first obesity prediction value may be expressed as a BMI.

The second prediction unit 132 may receive time-series physical information (including growth stage information, sex information, body composition information, and the like) of the evaluation subject from the input unit 11 to generate the second obesity prediction value, and for example, the second obesity prediction value may be expressed as a BMI. However, the second obesity prediction value may not only be expressed as the above-described BMI, but also may be expressed as a value in various forms based on various pieces of input information such as sex, growth stage, body composition information.

Meanwhile, the first prediction unit 131 and the second prediction unit 132 may independently receive the above-described information, or the second prediction unit 132 may receive the weight and height information of the evaluation subject through the first prediction unit 131.

As an example, the first prediction unit 131 may receive the height and weight information of the evaluation subject from the input unit 11 to generate the first prediction value, and the second prediction unit 132 may receive height, weight information, growth stage information, sex information, body composition information, and the like of the evaluation subject to generate the second prediction value. The second prediction unit 132 may generate the time-series physical information of the evaluation subject corresponding to the arbitrary period based on the time-series physical growth information of the plurality of sample subjects using the information of the evaluation subject input from the input unit 11 as described above.

As an example, the first prediction unit 131 may receive the height and weight information of the evaluation subject from the input unit 11 to generate the first prediction value, and the second prediction unit 132 may receive, from the input unit 11, information other than the height and weight information of the evaluation subject input to the first prediction unit to generate the second prediction value. The second prediction unit 132 may generate the time-series physical information of the evaluation subject corresponding to the arbitrary period based on the time-series physical growth information of the plurality of sample subjects using the information of the evaluation subject input from the input unit 11 as described above.

The second prediction unit 132 may classify the sex and the growth stage through the sex determination unit 15 and the growth stage determination unit 17 based on the physical information of the evaluation subject, extract physical information corresponding to the classified growth stage, and generate the second prediction value based on such information.

The third prediction unit 133 may generate the third prediction value based on the first prediction value and the second prediction value generated by the first prediction unit 131 and the second prediction unit 132, and the solution generation unit 19 may generate a solution based on the third prediction value in consideration of a current state, a predicted state, and the like of the evaluation subject, and display the generated solution through the display unit 12.

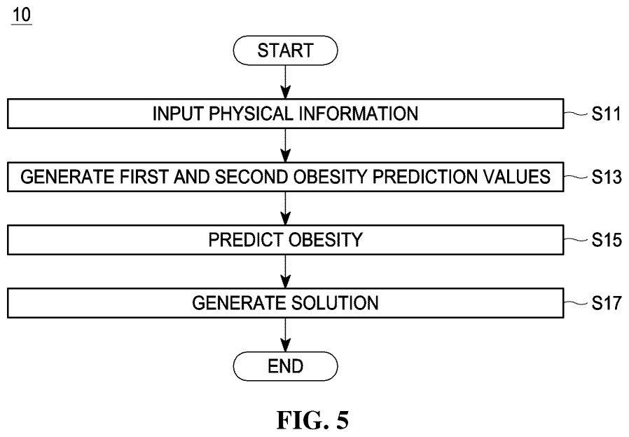

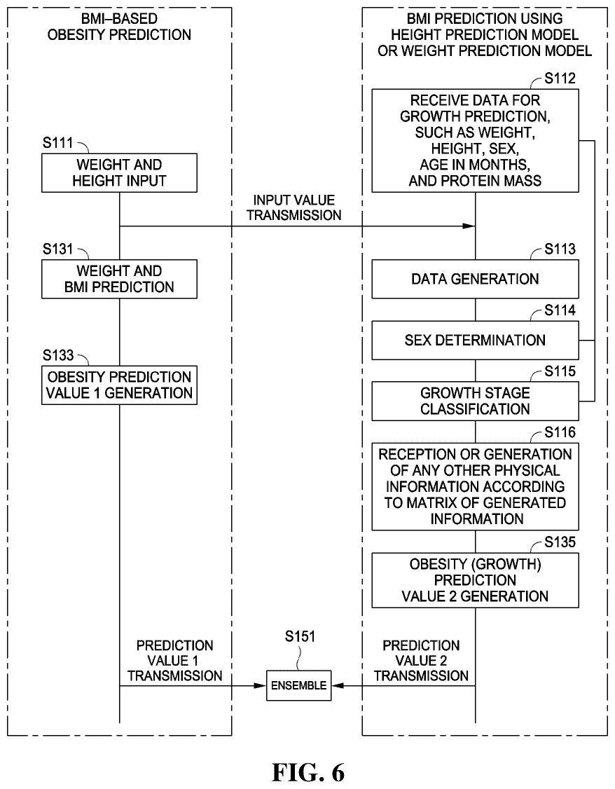

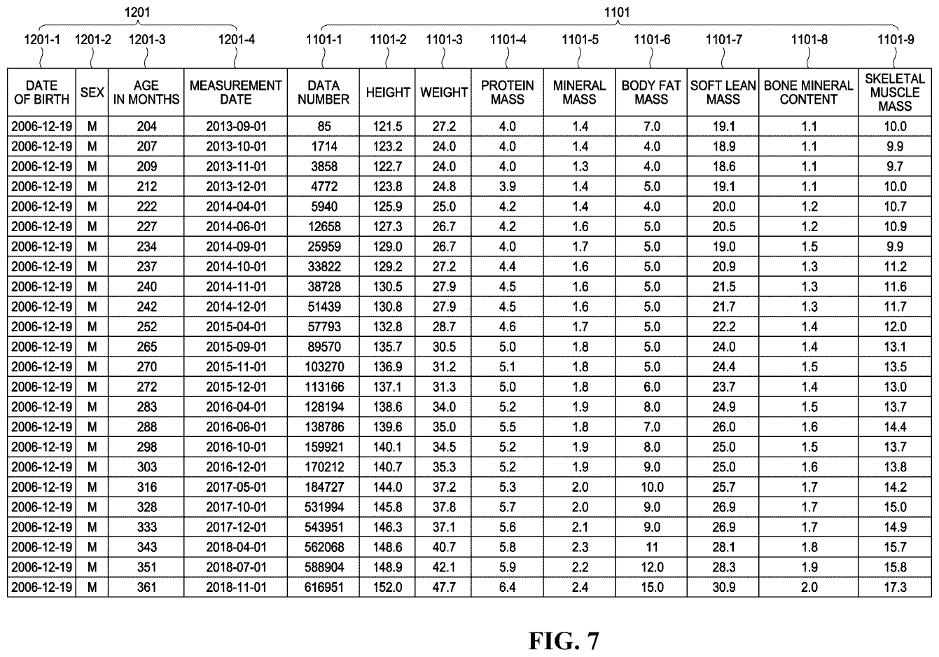

FIG. 5 is a diagram illustrating an obesity prediction method according to an exemplary embodiment of the present invention, FIG. 6 is a diagram illustrating a flow of generating a plurality of obesity prediction values according to an exemplary embodiment of the present invention, and FIG. 7 is a diagram illustrating identification information and body composition information of an evaluation subject according to an exemplary embodiment of the present invention.

Hereinafter, a description will be given with reference to FIGS. 5 to 7.

A method 10 of predicting obesity and providing a solution of the present embodiment may include: an operation S11 of receiving physical information of an evaluation subject; an operation S13 of generating a first obesity prediction value and a second obesity prediction value; an operation S15 of predicting obesity; and an operation S17 of generating a solution.

Operation S11 is an operation of receiving the physical information of the evaluation subject, and as described above, the first prediction unit 131 and the second prediction unit 132 may independently receive the physical information of the evaluation subject, the second prediction unit 132 may receive the physical information input to the first prediction unit 131, and if necessary, the second prediction unit 132 may receive remaining physical information from the input unit 11 or may generate the time-series physical information of the evaluation subject corresponding to the arbitrary period based on the time-series physical growth information of a plurality of sample subjects.

Operation S13 is an operation of generating a plurality of obesity prediction values through different neural networks trained based on the received physical information, and the first prediction value and the second prediction value may be generated through the first prediction unit 131 and the second prediction unit 132.

Operation S15 may predict the obesity of the evaluation subject by comparing (or generating the third prediction value) prediction values generated in operation S13 through the third prediction unit 133. As an example, in operation S13, the first prediction unit 131 may predict obesity through a BMI based on the weight and height information of the evaluation subject, and the second prediction unit 132 may predict obesity through a BMI based on a height prediction model or a weight prediction model.

Operation S17 may include generating a solution based on a result of the determination in operation S15.

First, generation of the first prediction value will be described.

The device 10 for predicting obesity using an ensemble model and providing a solution may receive the weight and height information of the evaluation subject through the input unit 11 (S111), and the first prediction unit 131 may predict the BMI of the evaluation subject based on the input weight and height information of the evaluation subject (S131) to generate the first prediction value (S133). In operation S131, a prediction point in time is preferably set to a point in time after the rapid growth stage among the growth stages. This is because the growth of the children and adolescents occurs within a large range in the rapid growth stage.

Hereinafter, generation of the second prediction value will be described.

The device 10 for predicting obesity using an ensemble model and providing a solution may receive information for growth prediction, such as weight, height, sex, age in months, protein mass, and growth stage through the input unit 11 (S112).

As described above, for the weight and height information, information input to the first prediction unit 131 may be received, and for remaining information, the time-series physical information of the evaluation subject corresponding to an arbitrary period may be generated based on the time-series physical growth information of the plurality of sample subjects (S113). It is obvious that sex, identification information, and the like may be received through the input unit 11, as needed.

Also, information necessary for generation of the second prediction value may be received independently from information input for generation of the first prediction value through the input unit 11 (S112), the time-series physical information of the evaluation subject corresponding to the arbitrary period may be generated based on the time-series physical growth information of the plurality of sample subjects (S113) for growth stage classification (S115), and any other physical information may be received or generated according to a matrix of the generated information (S116). Such generated information is illustrated as illustrated in FIG. 7.

The physical information of the evaluation subject may include information capable of identifying the subject (identification information) and biological component information of the subject.

As an example, the identification information may include information for identifying the subject, such as school grade (or age), sex, and height, and the biological component information may include information such as weight, protein, mineral content, body fat, body water, soft lean mass, body fat mass, bone tissue, skeletal muscle mass, BMI, basal metabolic rate, neck circumference, chest circumference, abdominal circumference, thigh circumference, arm circumference, and hip circumference.

More specifically, referring to FIG. 7, identification information 1201 of the subject may include date of birth 1201-1, sex 1201-2, age in months 1201-3, and measurement date 1201-4.

Also, biological component information 1101 of the subject may include data number 1101-1, height 1101-2, weight 1101-3, protein mass 1101-4, mineral mass 1101-4, body fat mass 1101-6, soft lean mass 1101-7, osseous mineral 1101-8, and skeletal muscle mass 1101-9.

Such physical information is merely an example for helping understanding of the present invention, the present embodiment is not limited thereto, and it is obvious that types of information constituting physical information may vary according to embodiments.

Meanwhile, an obesity (growth) prediction value (the second prediction value) may be generated based on the input information or generated information (S135).

The first prediction value and the second prediction value generated through the first prediction unit 131 and the second prediction unit 132 may be used to derive an ensemble-based obesity prediction result in the third prediction unit 133 (S151), and this will be described in more detail with reference to the drawings below.

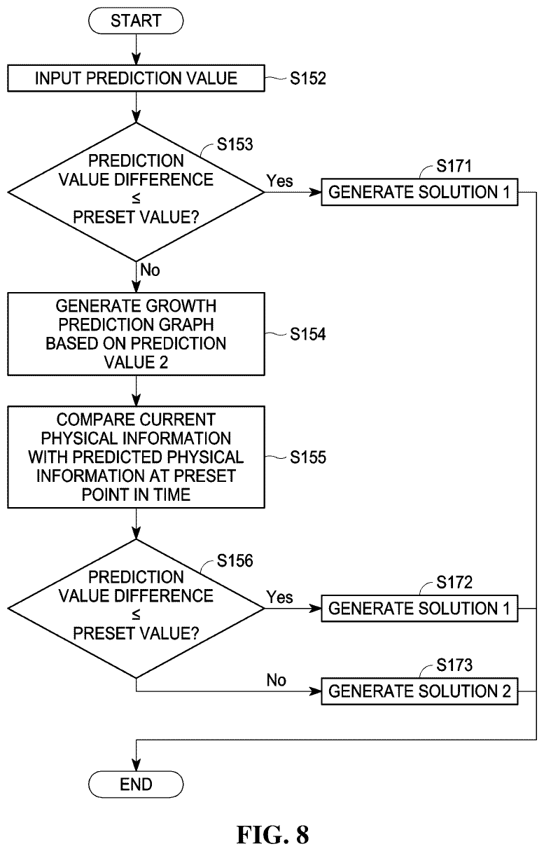

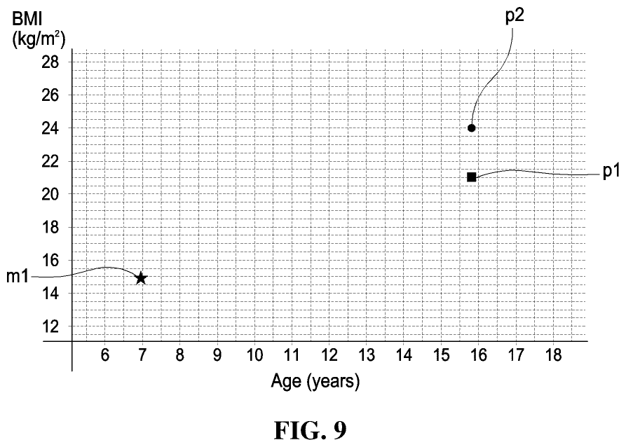

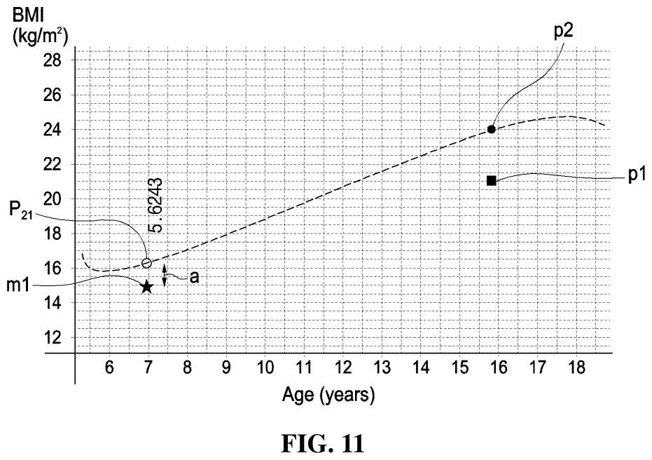

FIG. 8 is a diagram illustrating a solution providing method according to an exemplary embodiment of the present invention, and FIGS. 9 to 11 are diagrams expressing, on a graph, a method of predicting obesity of an evaluation subject and providing a solution according to an exemplary embodiment of the present invention.

Hereinafter, a description will be given with reference to FIGS. 8 to 11.

The device 10 for predicting obesity using an ensemble model and providing a solution according to an exemplary embodiment of the present invention may compare the first prediction value and the second prediction value generated in FIG. 6 through the third prediction unit 133, and generate different solutions according to a result of comparing the first prediction value and the second prediction value through the solution generation unit 19.

More specifically, the device 10 for predicting obesity using an ensemble model and providing a solution may receive the generated first prediction value and second prediction value (S152), and compare a difference between the first prediction value and the second prediction value with a preset value (S153).

When the difference between the first prediction value and the second prediction value is equal to or less than the preset value (S152: Yes), a first solution may be generated (S171). As an example, when the difference between the first prediction value and the second prediction value is equal to or less than the preset value (S152: Yes), the difference between the first prediction value and the second prediction value may be regarded as falling within an error range, and therefore, the evaluation subject may correspond to a case in which a probability of becoming obese at a prediction point in time (for example, any point in time after the rapid growth stage) is very high.

Accordingly, the first solution generated in operation S171 may generate a solution for improving factors directly related to obesity such as improving dietary habits, increasing physical activity, and improving lifestyle habits of the evaluation subject.

When the difference between the first prediction value and the second prediction value exceeds the preset value (S152: No), the third prediction unit 133 or the processor 14 may generate a growth prediction graph based on the second prediction value (S154), and compare current physical information with predicted physical information at a preset point in time to generate a solution (S155).

When the difference between the first prediction value and the second prediction value exceeds the preset value (S152: No), a determination may be made based on the second prediction value in which various factors are considered in order to increase the accuracy of an obesity prediction rate.

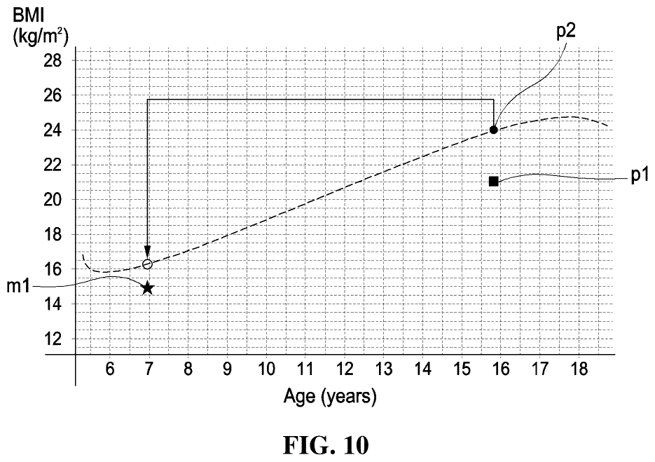

As an example, referring to FIG. 9, a BMI m1 of the evaluation subject measured at an evaluation point in time, a first prediction value p1, and a second prediction value p2 may be generated as age and BMI. When the difference (p1−p2) between the first prediction value and the second prediction value exceeds the preset value as described above, a growth prediction curve gc based on the second prediction value p2 may be generated as illustrated in FIG. 10 (S154).

The growth prediction curve gc may be generated based on time-series physical information of the plurality of sample subjects, or may be generated based on similar information extracted based on the physical information of the evaluation subject (S154).

Meanwhile, the third prediction unit 133 or the processor 14 derives an arbitrary prediction value p21 at a point at which the generated growth prediction curve gc intersects the preset point in time, and compares the derived arbitrary prediction value p21 with the physical information of the evaluation subject at the preset point in time (S155). The arbitrary prediction value p21 may be a BMI at the preset point in time on the growth prediction curve gc generated based on the second prediction value p2.

The preset point in time may be, for example, set as the evaluation point in time.

Also, the preset point in time may be set as a boundary point in time between a normal growth stage and a rapid growth stage. In this case, for the measured BMI m1 of the evaluation subject, a growth graph of children and adolescents or a growth prediction curve (not shown) based on the BMI m1 at the evaluation point in time may be generated and arbitrary prediction values at the preset point in time may be compared.

When a difference between the arbitrary prediction value p21 and the physical information m1 of the evaluation subject at the preset point in time is equal to or less than a preset value (S156: Yes), the solution generation unit 19 may generate a second solution (S172), and when the difference exceeds the preset value (S156: No), the solution generation unit 19 may generate a third solution (S173).

More specifically, when the difference between the arbitrary prediction value p21 and the physical information m1 of the evaluation subject at the preset point in time is equal to or less than the preset value (S156: Yes), the evaluation subject is highly likely to grow while showing a trend similar to the growth prediction curve gc generated based on the second prediction value p2.

Accordingly, the second solution generated through the solution generation unit 19 (S172) may include content for increasing a duration of the rapid growth stage among the growth stages. That is, various solutions for widening a range of the x-axis corresponding to the rapid growth stage 303 in FIG. 4 may be generated and provided to the evaluation subject through the display unit 12.

When the difference between the arbitrary prediction value p21 and the physical information m1 of the evaluation subject at the preset point in time exceeds the preset value (S156: No), the evaluation subject is likely to grow while showing features different from the growth prediction curve gc generated based on the second prediction value p2. The features may be a growth peak appearing in the rapid growth stage, and when the difference between the arbitrary prediction value p21 and the physical information m1 of the evaluation subject at the preset point in time exceeds the preset value (S156: No), a difference in the growth peak may occur.

Accordingly, the third solution generated through the solution generation unit 19 (S173) may include content for increasing a growth prediction value (growth peak) in the rapid growth stage among the growth stages. That is, various solutions for increasing a target value of the y-axis corresponding to the rapid growth stage 303 in FIG. 4 may be generated and provided to the evaluation subject through the display unit 12.

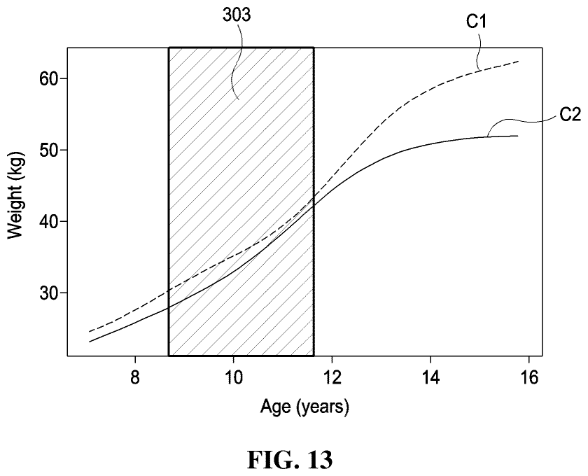

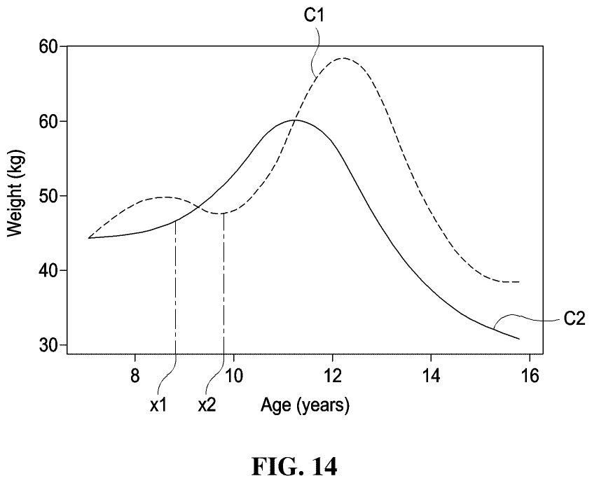

FIG. 12 is a diagram illustrating an obesity prediction method according to an exemplary embodiment of the present invention, FIG. 13 is a diagram illustrating a weight change by sex according to an exemplary embodiment of the present invention, and FIG. 14 is a diagram illustrating a weight change rate by sex for the same evaluation subject as in FIG. 13.

Hereinafter, a description will be given with reference to FIGS. 12 to 14.

A method S20 of predicting obesity and providing a solution of the present embodiment may include: an operation S21 of inputting a prediction value; an operation S23 of determining sex; an operation S25 of setting an obesity analysis point; an operation S27 of predicting obesity; and an operation S29 of generating a solution.

In the present embodiment, the first prediction value and the second prediction value generated in FIG. 6 described above through the third prediction unit 133 may be received (S21) or the second prediction value may be received, and the obesity analysis point according to the sex of the evaluation subject may be set so that obesity can be predicted and a solution can be generated.

More specifically, in FIGS. 13 and 14, a curve expressed as a dotted line indicates a growth curve c1 of a boy, and a curve expressed as a solid line indicates a growth curve c2 of a girl. It can be confirmed that weights of both the boy and the girl rapidly increase in the rapid growth stage 303.

However, there is a difference in point in time at which the boy and the girl enter the rapid growth stage 303, and referring to FIG. 14, the girl enters the rapid growth stage at a point in time x1, and the boy enters the rapid growth stage at a point in time x2.

Accordingly, according to the method S20 of predicting obesity and providing a solution of the present embodiment, the sex of the evaluation subject may be determined (S23) and an obesity analysis point may be set (S25), and in operation S25, analysis points in time may be set as points in time x1 and x2 at which the rapid growth stage starts, which are indicated in FIG. 14. In this case, the first prediction value may be compared with the second prediction value as described above with reference to FIG. 8, and the obesity analysis point may be set based on a preset reference (S25), so that obesity prediction can be performed (S27), or only the second prediction value may be input and compared with current physical information of the evaluation subject as described above with reference to FIG. 8, so that obesity prediction can be performed (S27).

For the comparison between the physical information of the evaluation subject and the second prediction value, as described above with reference to FIG. 8, the measured physical information of the evaluation subject and the growth prediction curve based on the second prediction value may be generated and then compared with each other at different reference points in time according to sex, to generate a solution (S29). For the growth prediction curve generated based on the physical information of the evaluation subject, a growth graph of children and adolescents may be utilized, or a method similar to the growth prediction curve based on the second prediction value may be utilized.

The solution generation unit 19 may generate a solution according to a result of the comparison between the physical information of the evaluation subject and the second prediction value (S29), and it is obvious that another solution according to the comparison result may be generated through a method similar to the above-described method.

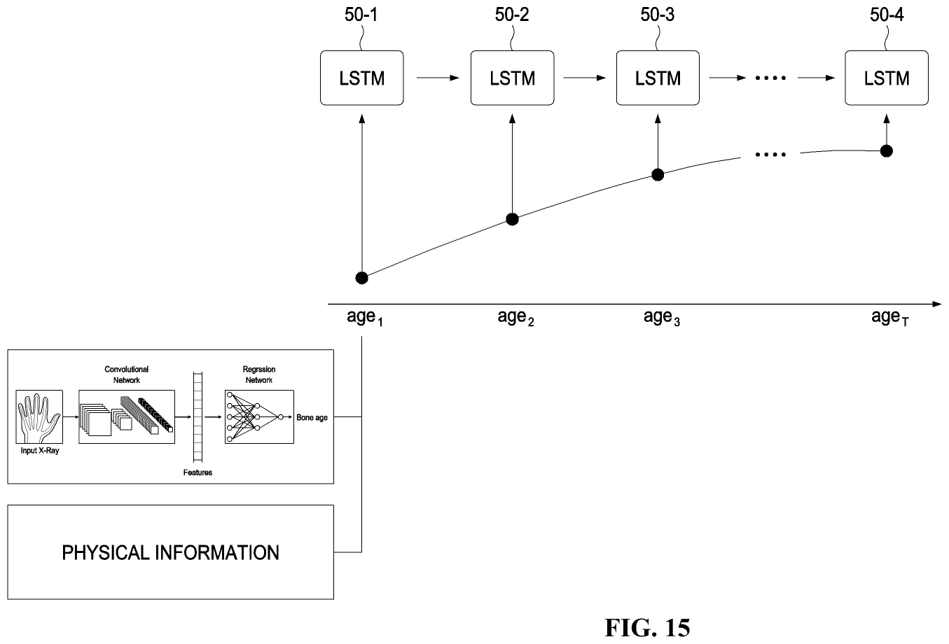

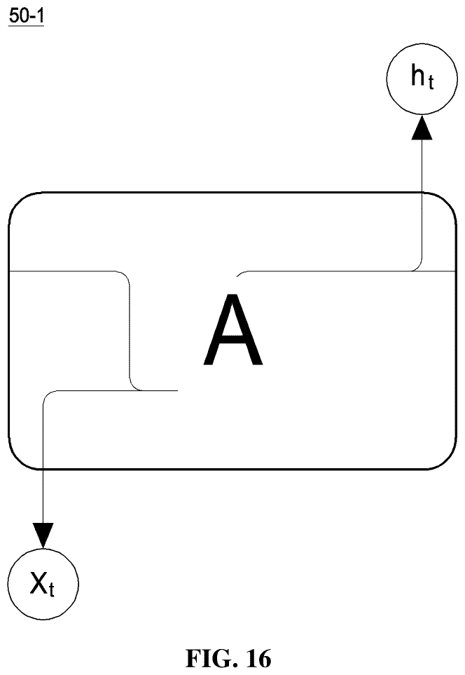

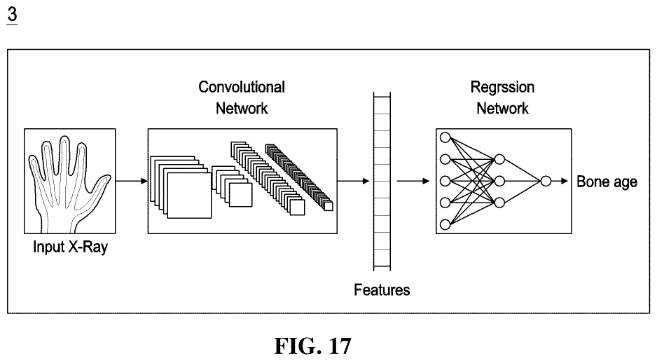

FIG. 15 is a diagram illustrating a configuration of a neural network according to an exemplary embodiment of the present invention, FIG. 16 is a diagram illustrating a first neural network model according to an exemplary embodiment of the present invention, and FIG. 17 is a diagram illustrating a second neural network model according to an exemplary embodiment of the present invention.

Hereinafter, a description will be given with reference to FIGS. 15 to 17.

According to an exemplary embodiment of the present invention, a first model 50 and a second model 13 may be included, and a pipeline in which at least a portion of output of the second model 13 is input to the first model 50 may be constructed.

More specifically, the first model 50 is a model that is trained by using, as training data, physical information corresponding to at least one of the plurality of growth stages based on the time-series physical information of the plurality of sample subjects.

As the growth stage, the rapid growth stage 303, which is a point in time at which growth reduction due to obesity occurs, may be adopted, but it is obvious that one or more of the plurality of growth stages can be adopted in order to predict obesity more accurately as described above.

The first model 50 includes LSTM neural networks 50-1, 50-2, 50-3, and 50-4 for time-series data learning, and trains the LSTM neural networks 50-1, 50-2, 50-3, and 50-4 using past physical information of the plurality of sample subjects. Then, the current physical information of the subject may be input to the trained LSTM neural networks 50-1, 50-2, 50-3, and 50-4, and predicted growth and a solution taking into account the growth may be output.

The physical information of the subject may be refined data subjected to an error determination process in a preprocessing unit.

The LSTM neural networks 50-1, 50-2, 50-3, and 50-4 are trained with at least one of the pieces of physical information of the plurality of sample subjects as a basic value. For example, for height, training is performed with annual height information during an arbitrary period or during a specific growth stage, and data is predicted for the next year and compared with actual data. With such a comparison, a training set moves to the future in units of the arbitrary period or specific growth stage and thus training is performed.

Also, the LSTM neural networks 50-1, 50-2, 50-3, and 50-4 may be trained for each growth stage. Accordingly, the LSTM neural networks may be trained with past physical information of the growth stages corresponding to the normal growth period 301, the rapid growth period 303, the decelerated growth period 305, and the growth plate closure period 307.

The physical information of the subject for learning may be refined data subjected to an error determination process in a preprocessing unit.

Meanwhile, in the present embodiment, the time-series physical information of the plurality of sample subjects may be sequentially input as training data according to age or an arbitrary period, and a result of calculating a prediction value at a past point in time or degree of growth is delivered to degree-of-growth prediction at the next age or arbitrary period.

Accordingly, the LSTM neural networks 50-1, 50-2, 50-3, and 50-4 may learn not only degree-of-growth prediction according to the current physical information, but also a degree to which prediction results 50-1, 50-2, 50-3, and 50-4 for various indicators at past points in time influence the current degree-of-growth prediction, and through this, extract, from the indicators, items having a large influence on change in degree of growth according to age or an arbitrary period, and reflect the items in degree-of-growth prediction.

Also, for time-series learning, it is necessary to secure the physical information of the plurality of sample subjects at regular times. However, as described above, it may be difficult to acquire the physical information of the plurality of sample subjects regularly according to age or an arbitrary period, and therefore, outlier physical information for each unit period or non-continuous physical information may be removed for temporal normalization and used.

Meanwhile, the second model 13 may derive bone maturity (age) from a hand-wrist bone image using a convolutional neural network trained with bone maturity information of the subject as training data.

More specifically, the convolutional neural network includes a large number of convolution layers that create feature maps for features in an analysis target image among hand-wrist bone images, and pooling layers in which sub-sampling is performed between the large number of convolution layers, thereby extracting different levels of features for an analysis target region, and the features may be probabilistically inferred through an activation function or the bone maturity may be derived through weighted learning between nodes using regression analysis.

The bone maturity degree derived through the second model 13 may be input to the LSTM neural networks 50-1, 50-2, 50-3, and 50-4 together with at least a portion of the physical information of the subject, thereby increasing the accuracy in predicting the growth of the subject and further increasing the accuracy of the obesity prediction.

The present invention has been described above focusing on preferred embodiments thereof. All embodiments and conditional examples disclosed in the present specification have been described with an intention to help those skilled in the art of the present invention understand the principles and concepts of the present invention, and it will be understood by those skilled in the art that the present invention may be implemented in modified forms without departing from essential characteristics of the present invention.

Accordingly, the disclosed embodiments should be considered from a descriptive viewpoint rather than a limiting viewpoint. The scope of the present invention is shown in the claims rather than the above description, and all differences within an equivalent scope should be construed as being included in the present invention.

Meanwhile, the methods according to various embodiments of the present invention described above may be implemented as programs and provided to a server or device. Accordingly, each device may access the server or the device in which the program is stored, and download the program.

Also, the above-described methods according to various embodiments of the present invention may be implemented as programs and stored and provided in various non-transitory computer readable media. The non-transitory computer-readable medium is a medium that stores data semi-permanently and is readable by a device, not a medium that stores data for a short period of time such as a register, a cache, or a memory. Specifically, the various applications or programs described above may be stored and provided in a non-transitory computer-readable medium such as a CD, a DVD, a hard disk, a Blu-ray disc, a USB, a memory card, or a ROM.

Also, although preferred embodiments of the present invention have been illustrated and described above, the present invention is not limited to the specific embodiments described above, it is obvious that various modified embodiments may be made by those skilled in the art without departing from the gist of the present invention defined in the claims, and such modified embodiments should not be understood individually from the technical idea or perspective of the present invention.

1. A method of predicting obesity using an ensemble technique, comprising:
receiving physical information of an evaluation subject;
generating a plurality of obesity prediction values through different neural networks trained based on the received physical information;
predicting obesity by comparing the plurality of obesity prediction values; and
generating different solutions according to a difference between the plurality of obesity prediction values.

2. The method of claim 1, wherein the plurality of obesity prediction values include:
a first obesity prediction value generated based on weight and height information of the evaluation subject; and
a second obesity prediction value generated based on information including growth stage information of the evaluation subject.

3. The method of claim 2, wherein the physical information of the evaluation subject is received to generate the first obesity prediction value and the second obesity prediction value.

4. The method of claim 2, wherein the second obesity prediction value is generated based on information further including the weight and height information of the evaluation subject.

5. The method of claim 4, wherein the second obesity prediction value is generated based on information including the weight and height information of the evaluation subject input to generate the first obesity prediction value.

6. The method of claim 2, wherein the generating of the solutions includes:
receiving the first obesity prediction value and the second obesity prediction value; and
comparing a difference between the first obesity prediction value and the second obesity prediction value with a preset reference.

7. The method of claim 6, wherein the generating of the solutions further includes a first solution generation step of generating a solution for reducing a weight of the evaluation subject when a difference between the first obesity prediction value and the second obesity prediction value is equal to or less than a preset value in the comparison step.

8. The method of claim 6, wherein the generating of the solutions further includes generating a growth graph based on the second obesity prediction value when a difference between the first obesity prediction value and the second obesity prediction value exceeds a preset value in the comparison step.

9. The method of claim 8, wherein the generating of the solutions further includes a second solution generation step of comparing information on the growth graph generated based on the second obesity prediction value with the received physical information of the evaluation subject to generate a solution.

10. The method of claim 9, wherein the second solution generation step includes comparing the growth graph generated based on the second obesity prediction value with the physical information of the evaluation subject at a point in time at which the physical information of the evaluation subject is input.

11. The method of claim 10, wherein the second solution generation step includes providing a solution for increasing a growth prediction value in a rapid growth stage among growth stages when a difference between the information on the growth graph generated based on the second obesity prediction value and the physical information of the evaluation subject exceeds a preset value.

12. The method of claim 10, wherein the second solution generation step includes providing a solution for increasing a duration of a rapid growth stage among growth stages when the difference between the information on the growth graph generated based on the second obesity prediction value and the physical information of the evaluation subject is equal to or less than a preset value.

13. The method of claim 9, wherein, in the second solution generation step, the physical information of the evaluation subject that is a comparison target is a body mass index.

14. A program stored in a computer-readable recording medium, the program including program code for executing the method of predicting obesity using an ensemble technique according to claim 1.

15. A computer-readable recording medium including program code for executing the method of predicting obesity using an ensemble technique according to claim 1.

16. A device for predicting obesity using an ensemble technique and providing a solution, comprising:
an input unit configured to receive physical information of an evaluation subject;
an obesity prediction unit configured to generate a plurality of obesity prediction values based on the physical information of the evaluation subject input to the input unit; and
a solution generation unit configured to generate an obesity management solution based on the physical information of the evaluation subject when the evaluation subject corresponds to obese,
wherein the solution generation unit generates different solutions according to a difference between the plurality of obesity prediction values.

17. The device of claim 16, wherein the obesity prediction unit includes:
a first prediction unit configured to generate a first obesity prediction value based on weight and height information of the evaluation subject; and
a second prediction unit configured to generate a second obesity prediction value based on information including growth stage information of the evaluation subject.

18. The device of claim 17, wherein the obesity prediction unit further includes a third prediction unit configured to generate an obesity prediction value based on an ensemble of the first prediction unit and the second prediction unit.

19. The device of claim 18, wherein the third prediction unit calculates a difference between the first obesity prediction value and the second obesity prediction value.

20. The device of claim 17, wherein the solution generation unit compares a difference between the first obesity prediction value and the second obesity prediction value with a preset reference to generate different solutions.
